(19) United States
(12) Patent Application Publication
(10) Pub. No.: US 2020/0407720 A1
(43) Pub. Date: Dec. 31, 2020

(54) A DBAIT MOLECULE AGAINST ACQUIRED RESISTANCE IN THE TREATMENT OF CANCER

(71) Applicants: ONXEO, PARIS (FR); INSTITUT CURIE, PARIS CEDEX 05 (FR); INSERM (INSTITUT NATIONAL DE LA SANTÉ ET DE LA RECHERCHE MÉDICALE), PARIS (FR); CENTRE NATIONAL DE LA RECHERCHE SCIENTIFIQUE, PARIS (FR)

(72) Inventors: FRANÇOISE BONO, TOULOUSE (FR); WAEL JDEY, LONGJUMEAU (FR); MARIE DUTREIX, L'HAY-LES-ROSES (FR)

(21) Appl. No.: 16/979,892

(22) PCT Filed: Mar. 12, 2019
(86) PCT No.: PCT/EP2019/056077

§ 371 Date: Sep. 11, 2020

(30) Foreign Application Priority Data
Mar. 13, 2018 (EP) 18161607.9

(51) Int. Cl. C12N 15/113; A61K 45/06; A61P 35/00

(52) U.S. Cl. CPC C12N 15/113; A61K 45/06; C12N 2310/351; C12N 2310/14; A61P 35/00

(57) ABSTRACT

The invention relates to a method for delaying and/or preventing development of cancer resistant to a cancer therapy agent in a patient based on administration of a Dbait molecule.

### FIELD OF THE INVENTION

The present invention relates to the field of medicine, in particular of oncology.

### BACKGROUND OF THE INVENTION

While some patients achieve complete response and complete remission, a majority of the patients achieve moderate and poor responses to conventional therapies such as radiotherapy and/or chemotherapy and targeted therapies; the disease relapses in a significant majority of the patients. Relapse also occurs for certain number of patients following these therapies. Attempts to treat some relapsed patients with maintenance therapy, whether treated with such conventional therapies, have been met with limited success.

The relatively rapid acquisition of resistance to cancer drugs remains a key obstacle to successful cancer therapy. While some specific resistance-conferring mutations have indeed been identified in many cancer patients demonstrating acquired drug resistance, the relative contribution of mutational and non-mutational mechanisms to drug resistance, and the role of tumor cell subpopulations remain somewhat unclear.

New treatment methods are needed to successfully address heterogeneity within cancer cell populations and the emergence of cancer cells resistant to therapies.

### SUMMARY OF THE INVENTION

The present invention relates to a Dbait molecule for use in delaying and/or preventing development of cancer resistant to a cancer therapy agent in a patient.

The inventors have indeed shown that administration of repeated doses of a Dbait molecule, e.g. at least one dose of AsiDNA over several cycles of treatment, has the following advantages:

i) tumor cells did not become resistant to a Dbait molecule after repeated treatments;

ii) tumor cells did not become resistant to a cancer therapy agent such as a PARP inhibitor and a chemotherapy such as platinum agents;

ii) tumor cells became more sensitive to a Dbait molecule after each cycle of treatment; and

iii) non-tumor cells were not affected (no toxicity) by repeated treatments.

The present disclosure also provides a novel maintenance therapy regimen for the treatment of cancer in both monotherapy and combination therapy.

When a Dbait molecule is administered in combination with a cancer therapy (targeted therapy or not), it increases the period of cancer sensitivity and/or delays and/or prevents development of cancer resistance to the combined cancer therapy agent, preferably when both molecules are administered concomitantly or simultaneously. The inventors have thus shown that repeated treatments allowed the emergence of resistance to a cancer therapy agent, which is prevented and even reversed in presence of a Dbait molecule such as AsiDNA, indicating an unlikely tumor escape to this combined therapy.

These advantages lead amongst others to a reduced tumor recurrence (since it results that a Dbait molecule may be administered over a prolonged period of time). Accordingly, with the repeated administration of a Dbait molecule such as AsiDNA, the risk of recurrence found with the use of conventional and targeted anticancer is thus considerably limited.

In a first aspect, the cancer therapy agent is a Dbait molecule. In a second aspect, the cancer therapy agent is a chemotherapy or a targeted therapy. For instance, the cancer therapy agent can be a PARP inhibitor such as olaparib, rucaparib, niraparib, talazoparib, iniparib and veliparib. Alternatively, the cancer therapy agent can be selected from the group consisting of a platinum agent, an alkylating agent, a camptothecin, a nitrogen mustard, an antibiotic, an antimetabolite, and a vinca, preferably a platinum agent such as cisplatin, oxaliplatin and carboplatin.

Preferably, the Dbait molecule is to be administered by repeated administration, preferably for at least two cycles of administration. In particular, the Dbait molecule is to be administered in combination therapy with the cancer therapy agent, preferably for at least two cycles of administration, more preferably for at least three or four cycles of administration.

The present invention thus further relates to a Dbait molecule for use in a maintenance therapy for cancer treatments.

This therapy regimen may follow an induction therapy with for instance a conventional cancer therapy such as radiotherapy and/or chemotherapy or with a targeted therapy.

The present invention also relates to a Dbait molecule for use in treating a patient with cancer who is resistant or has increased likelihood of developing resistance to a cancer therapy agent. Preferably, the Dbait molecule has at least one free end and a DNA double stranded portion of 20-200 bp with less than 60% sequence identity to any gene in a human genome.

More preferably, the Dbait molecule has one of the following formulae:

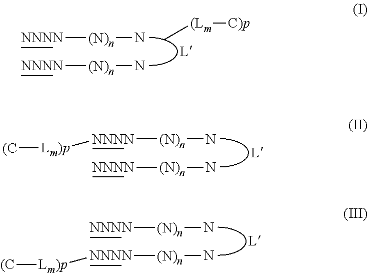

wherein N is a deoxynucleotide, n is an integer from 1 to 195, the underlined N refers to a nucleotide having or not a modified phosphodiester backbone, L′ is a linker, C is a molecule facilitating endocytosis preferably selected from a lipophilic molecule and a ligand which targets cell receptor enabling receptor mediated endocytosis, L is a linker, m and p, independently, are an integer being 0 or 1.

In a very specific embodiment, the Dbait molecule has the following formula (AsiDNA):

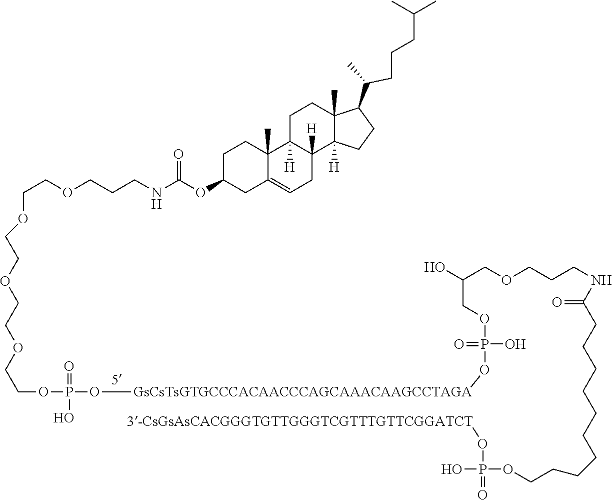

All cancer type can be treated. More preferably, the cancer is selected from leukemia, lymphoma, sarcoma, melanoma, and cancers of the head and neck, kidney, ovary, pancreas, prostate, thyroid, lung, in particular small-cell lung cancer, and non-small-cell lung cancer, esophagus, breast (including TBNC), bladder, colorectum, liver, cervix, and endometrial and peritoneal cancers. In particular, the cancer is a solid cancer.

### BRIEF DESCRIPTION OF THE FIGURES

FIG. 1. Repeated cycles of AsiDNA treatment didn't recover resistance.

(A) Protocol of repeated treatments with AsiDNA (504. Black arrow, cell survival assessment, drug removal and amplification of surviving cells; grey arrow, cell counting, freezing and seeding for the next treatment cycle. (B) Efficacy of AsiDNA (one to four cycles) on MDA-MB-231 cell line. Cell survival was calculated as the ratio of living treated cells/living not-treated cells. (C) Growth of MDA-MD-231 cell-derived xenografted tumors mock treated (Black lines) or treated (grey lines) with three cycles of AsiDNA (grey rectangles) according to the protocol five consecutive days injections followed by 17 days rest (vehicle, n=6; AsiDNA, n=8). Data are represented as mean±s.e.m.

FIG. 2. AsiDNA abrogates the emergence of resistance to PARP inhibitors. (A) Protocol of drug repeated treatments. Black arrow, cell survival assessment, drug removal and amplification of surviving cells; grey arrow, cell counting, freezing and seeding for the next treatment cycle. (B) Assessment of the effect of AsiDNA (2.504) co-treatment on the emergence of resistance to PARP inhibitors olaparib (10 μM) and talazoparib (100 nM), according to treatment protocol presented in A.

FIG. 3. AsiDNA reverses the acquired resistance to the PARP inhibitor talazoparib. (A) Protocol of talazoparib resistance induction followed by i) resistance validation or ii) reversion with the combined treatment talazoparib+AsiDNA. (B) Cell survival was calculated as the ratio of living treated cells to living not-treated cells. RT, Resistant to Talazoparib.

FIG. 4. AsiDNA abrogates the emergence of resistance to niraparib in ovarian cancer. (A) Protocol of drug repeated treatments. Black arrow, cell survival assessment, drug removal and amplification of surviving cells; grey arrow, cell counting, freezing and seeding for the next treatment cycle. (B) Assessment of the effect of AsiDNA (2.504) co-treatment on the emergence of resistance to niraparib (504), according to treatment protocol presented in A.

FIG. 5. AsiDNA abrogates the emergence of resistance to talazoparib in SCLC. (A) Protocol of drug repeated treatments. Black arrow, cell survival assessment, drug removal and amplification of surviving cells; grey arrow, cell counting, freezing and seeding for the next treatment cycle. (B) Assessment of the effect of AsiDNA (2.5 μM) co-treatment on the emergence of resistance to talazoparib (100 nM), according to treatment protocol presented in A.

FIG. 6. AsiDNA abrogates the emergence of resistance to carboplatin in SCLC. (A) Protocol of drug repeated treatments. Black arrow, cell survival assessment, drug removal and amplification of surviving cells; grey arrow, cell counting, freezing and seeding for the next treatment cycle. (B) Assessment of the effect of AsiDNA (2.5 μM) co-treatment on the emergence of resistance to carboplatin (2.5 μM), according to treatment protocol presented in A.

### DETAILED DESCRIPTION OF THE INVENTION

In a first aspect, the present invention relates to a Dbait molecule for use in delaying and/or preventing development of cancer resistant to a cancer therapy agent in a patient. It also relates to a composition comprising a Dbait molecule for use in delaying and/or preventing development of cancer resistant to a cancer therapy agent in a patient; or to the use of a Dbait molecule for the manufacture of a drug for use in delaying and/or preventing development of cancer resistant to a cancer therapy agent in a patient. It further relates to a method for delaying and/or preventing development of cancer resistant to a cancer therapy agent in a patient, comprising administering an effective amount of a Dbait molecule, thereby delaying and/or preventing development of cancer resistant to a cancer therapy agent. More particularly, the method comprises administering an effective amount of a cancer therapy agent and administering an effective amount of a Dbait molecule, thereby delaying and/or preventing development of cancer resistant to a cancer therapy agent.

In another aspect, the present disclosure relates to a Dbait molecule for use in a maintenance therapy for cancer.

As used herein, the term “maintenance therapy” refers to a therapy, therapeutic regimen or course of therapy which is administered subsequent to an induction therapy (an initial course of therapy administered to an individual or subject with a disease or disorder). Maintenance therapy can be used to halt or reverse the progression of the disease/disorder), to maintain the improvement in health achieved by induction therapy and/or enhance, or “consolidate”, the gains obtained by induction therapy. Accordingly, maintenance therapy is mainly used to prevent or minimize the risk of disease relapse.

A therapy, in particular the maintenance therapy, may employ continuous therapy (e.g., administering a drug at a regular interval, (e.g., weekly, monthly, yearly, etc.) or intermittent therapy (e.g., interrupted treatment, intermittent treatment, treatment at relapse, or treatment upon achievement of a particular predetermined criteria (e.g., disease manifestation, etc.).

In one embodiment, the therapy, in particular the maintenance therapy, consists of a repeated administration regimen.

As used herein, the term “repeated administration” refers to the drug administration of a fixed dose at a regular time interval of a drug. In repeated administration, accumulation occurs when the drug is administered before the previous dose is completely eliminated. As a result of accumulation, plasma concentrations reach higher levels during repeated regimen than after administration of a single dose. The repeated administration regimen is used to ensure an exposure to the drug within the therapeutic range over a prolonged time. Accordingly, the Dbait molecule may be administered at intervals of, e.g., daily, twice per week, three times per week, or one time each week, two weeks, three weeks, monthly . . .

In some embodiments, the steady state plasma concentration must be reached more rapidly. A higher dose (also known as initial dose or loading dose) may then be administered on treatment initiation, to compensate for accumulation. Then, in some embodiments, “repeated doses” mean the administration of at least one particular dose of Dbait molecule after an initial higher dose. The treatment includes several cycles, for instance two to ten cycles, in particular two, three, four or five cycles. The cycles may be continued or separated. For instance, each cycle is separated by a period of time of one to eight weeks.

In one embodiment, the Dbait molecule is administered in monotherapy (as a stand-alone therapeutic regimen). In some embodiments, a Dbait molecule was used during the induction therapy. In a preferred embodiment, the Dbait molecule is AsiDNA. In other embodiment, the Dbait molecule is administered in combination therapy with a cancer therapy agent. In some embodiments, the cancer therapy agent is the agent used during the induction therapy. In other embodiments, the cancer therapy agent is not the agent used during the induction therapy.

In some embodiments, “combination therapy” is intended to embrace administration of these therapeutic agents in a sequential manner, wherein each therapeutic agent is administered at a different time, as well as administration of these therapeutic agents, or at least two of the therapeutic agents concurrently, or in a substantially simultaneous manner. Preferably, the Dbait molecule and the cancer therapy agent are administered concomitantly or simultaneously. The term “concomitantly” is used herein to refer to administration of two or more therapeutic agents, give in close enough temporal proximity where their individual therapeutic effects overlap in time. Accordingly, concurrent administration includes a dosing regimen when the administration of one or more agent(s) continues after discontinuing the administration of one or more other agent(s).

As used herein, and unless otherwise specified, the term “in combination with” includes the administration of two or more therapeutic agents simultaneously, concomitantly, or sequentially within no specific time limits unless otherwise indicated. In one embodiment, a Dbait molecule is administered in combination with a cancer therapy agent (chemotherapy, radiotherapy, targeted therapy such as a PARP inhibitor). In one embodiment, the agents are present in the cell or in the subject's body at the same time or exert their biological or therapeutic effect at the same time. In certain embodiments, a first agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), essentially concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, or any combination thereof. For example, in one embodiment, the first agent can be administered prior to the second therapeutic agent, for e.g. 1 week. In another, the first agent can be administered prior to (for example 1 day prior) and then concomitant with the second therapeutic agent.

The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Therapeutic agents may also be administered in alternation. In certain embodiments the therapeutically effective amount of each agent used in combination will be lower when used in combination in comparison to monotherapy with each agent alone. Such lower therapeutically effective amount could afford for lower toxicity of the therapeutic regimen.

In some embodiments, the cancer therapy agent is chemotherapy. As used herein, the term “chemotherapy” refers to a chemical compound useful in the treatment of cancer.

Examples of chemotherapies include alkylating agents such as cyclosphosphamide (CYTOXAN®); bisphosphonates such as clodronate (BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELK)®) and risedronate (ACTONEL®); a camptothecin (including the synthetic analogue topotecan (HYCAMTIN®) and CPT-11 (irinotecan, CAMPTOSAR®)), nitrogen mustards such as chlorambucil and melphalan; antibiotics such doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), and peglylated liposomal doxorubicin (CAELYX®); anti-metabolites such as methotrexate, gemcitabine (GEMZAR®), tegafur (UFTORAL®), capecitabine (XELODA®) and 5-fluorouracil (5-FU); taxoid, e.g., paclitaxel (TAXOL®), albumin-engineered nanoparticle formulation of paclitaxel (ABRAXANE™), and docetaxel (TAXOTERE®); platinum agents such as cisplatin, oxaliplatin (ELOXATIN®), and carboplatin; vincas, which prevent tubulin polymerization from forming microtubules, including vinblastine (VELBAN®), vincristine (ONCOVIN®), vindesine (ELDISINE®, FILDESIN®) and vinorelbine (NAVELBINE®). In some embodiments, the chemotherapy is a platinum agent. In some embodiments, the platinum agent is cisplatin. In some embodiments, the platinum agent is oxaliplatin. In some embodiments, the platinum agent is carboplatin.

In some embodiments, the cancer therapy agent is radiotherapy.

In other embodiments, the cancer therapy agent is a targeted therapy. As used herein, the term “targeted therapy” refers to a therapeutic agent that binds to polypeptide(s) of interest and inhibits the activity and/or activation of the specific polypeptide(s) of interest.

Examples of such agents include antibodies and small molecules that bind to the polypeptide of interest. The targeted therapy may be a PARP inhibitor (such as olaparib (LYNPARZA®), rucaparib (RUBRACA®), niraparib (ZEJULA®), talazoparib, iniparib and veliparib), which binds and inhibits Poly (ADP-ribose) polymerase (PARP).

Parp Inhibitors:

Provided here are also PARP inhibitors useful in the methods described herein. PARP is meant Poly (ADP-ribose) polymerase. PARP catalyzes the conversion of β-nicotinamide adenine dinucleotide (NAD+) into nicotinamide and poly-ADP-ribose (PAR). PARP is a key molecule in the repair of DNA single-strand breaks (SSBs). As used herein, the term “PARP inhibitor” refers to any compound which has the ability to decrease the activity of a poly (ADP-ribose) polymerase (PARP). PARP inhibition relies mainly on two different mechanisms: (i) catalytic inhibition that act mainly by inhibiting PARP enzyme activity and (ii) bound inhibition that block PARP enzyme activity and prevent its release from the damage site. Bound inhibitors are more toxic to cells than catalytic inhibitors. PARP inhibitors according to the inventions are preferably catalytic and/or bound inhibitors. Many PARP inhibitors are known and, thus, can be synthesized by known methods from starting materials that are known, may be available commercially, or may be prepared by methods described in the literature.

Examples of suitable PARP inhibitors according to the invention include, but are not limited to, olaparib (AZD-2281, 4-[(3-[(4-cyclopropylcarbonyl)piperazin-4-yl]carbonyl)-4-fluorophenyl]methyl(2H)-phthalazin-1-one), veliparib (ABT-888, CAS 912444-00-9, 2-((fi)-2-methylpyrrolidin-2-yl)-1W-benzimidazole-4-carboxamide), CEP-8983 (ll-methoxy-4,5,6,7-tetrahydro-1H-cyclopenta[a]pyrrolo[3,4-c]carbazole-1,3(2H)-dione) or a prodrug thereof (e.g. CEP-9722), rucaparib (AG014699, PF-01367338, 8-Fluoro-2-{4-[(methylamino)methyl]phenyl}-1,3,4,5-tetrahydro-6H-azepino[5,4,3-cd]indol-6-one), E7016 (GPI-21016, 10-((4-Hydroxypiperidin-1-yl)methyl)chromeno-[4,3,2-de]phthalazin-3 (2H)-one), talazoparib (BMN-673, (8S,9R)-5-fluoro-8-(4-fluorophenyl)-9-(1-methyl-1H-1,2,4-triazol-5-yl)-8,9-dihydro-2H-pyrido[4,3,2de]phthalazin-3(7H)-one), INO-1001 (4-phenoxy-3-pyrrolidin-1-yl-5-sulfamoyl-benzoic acid), KU0058684 (CAS 623578-11-0), niraparib (MK 4827, Merck & Co Inc), iniparib (BSI 201), iniparib-met (C-nitroso metabolite of Iniparib), CEP 9722 (Cephalon Inc), LT-673, MP-124, NMS-P118, XAV939 and AZD 2461. Additional PARP inhibitors are described for example in WO14201972, WO14201972, WO12141990, WO10091140, WO9524379, WO09155402, WO09046205, WO08146035, WO08015429, WO0191796, WO0042040, US2006004028, EP2604610, EP1802578, CN104140426, CN104003979, U.S. Ser. No. 06/022,9351, U.S. Pat. No. 7,041,675, WO07041357, WO2003057699, U.S. Ser. No. 06/444,676, US20060229289, US20060063926, WO2006033006, WO2006033007, WO03051879, WO2004108723, WO2006066172, WO2006078503, US20070032489, WO2005023246, WO2005097750, WO2005123687, WO2005097750, U.S. Pat. Nos. 7,087,637, 6,903,101, WO20070011962, US20070015814, WO2006135873, UA20070072912, WO2006065392, WO2005012305, WO2005012305, EP412848, EP453210, EP454831, EP879820, EP879820, WO030805, WO03007959, U.S. Pat. No. 6,989,388, EP1212328, WO2006078711, U.S. Ser. No. 06/426,415, U.S. Ser. No. 06/514,983, EP1212328, US20040254372, US20050148575, US20060003987, U.S. Ser. No. 06/635,642, WO200116137, WO2004105700, WO03057145A2, WO2006078711, WO2002044157, US20056924284, WO2005112935, US20046828319, WO2005054201, WO2005054209, WO2005054210, WO2005058843, WO2006003146, WO2006003147, WO2006003148, WO2006003150, WO2006003146, WO2006003147, UA20070072842, U.S. Ser. No. 05/587,384, US20060094743, WO2002094790, WO2004048339, EP1582520, US20060004028, WO2005108400, U.S. Pat. No. 6,964,960, WO20050080096, WO2006137510, UA20070072841, WO2004087713, WO2006046035, WO2006008119, WO06008118, WO2006042638, US20060229289, US20060229351, WO2005023800, WO1991007404, WO2000042025, WO2004096779, U.S. Ser. No. 06/426,415, WO02068407, U.S. Ser. No. 06/476,048, WO2001090077, WO2001085687, WO2001085686, WO2001079184, WO2001057038, WO2001023390, WO01021615A1, WO2001016136, WO2001012199, WO95024379, WO200236576, WO2004080976, WO2007149451, WO2006110816, WO2007113596, WO2007138351, WO2007144652, WO2007144639, WO2007138351, WO2007144637.

In a preferred embodiment, the PARP inhibitor is selected from the group consisting of rucaparib (AG014699, PF-01367338), olaparib (AZD2281), veliparib (ABT888), iniparib (BSI 201), niraparib (MK 4827), talazoparib (BMN673), AZD 2461, CEP 9722, E7016, INO-1001, LT-673, MP-124, NMS-P118 and XAV939.

In another aspect, the present invention relates to a Dbait molecule for use in increasing progression-free survival (PFS) in a patient. As used herein, the term “Progression-free survival” or “PFS” refers to the time (in years) measured from the start of maintenance therapy during which the disease being treated does not worsen. Progression-free survival is a metric that denotes the chances of a disease stabilizing or being reversed in a group of individuals suffering from the disease. For instance, it denotes the percentage of individuals in the group who are likely to be as healthy if not healthier after a particular period of time following the start of maintenance therapy. In some embodiments, the patient is a patient with cancer who is resistant or has increased likelihood of developing resistance to a cancer therapy agent commonly used for treating said cancer.

In a further aspect, the present disclosure relates to a Dbait molecule for use in a long-term duration therapy for cancer. A Dbait molecule is administered over a prolonged period of time accordingly. By a “prolonged period of time” is meant several months (for instance over 3, 6, 9 or 12 months and even several years (for instance over 1, 2 or 3 years). The present invention relates to a method of treating cancer in a patient comprising administering an effective amount of a Dbait molecule over a prolonged period of time. In some embodiments, the patient is a patient with cancer who is resistant or has increased likelihood of developing resistance to a cancer therapy agent commonly used for treating said cancer.

In an additional aspect, the present invention relates to a Dbait molecule for use in increasing relapse-free survival (RFS) in a patient. As used herein, the term “Relapse-free survival” or “RFS” refers to the time (in years) measured from diagnosis to first recurrence of the disease, e.g., first recurrence of a malignancy in a neoplastic disease. RFS is defined only for patients achieving complete remission, and is measured from the date of achievement of a remission until the date of relapse or death from any cause. In some embodiments, the patient is a patient with cancer who is resistant or has increased likelihood of developing resistance to a cancer therapy agent commonly used for treating said cancer.

In another aspect, the present invention relates to Dbait molecule for use in preventing or reducing tumor recurrence in a patient. In some embodiments, the patient is a patient with cancer who is resistant or has increased likelihood of developing resistance to a cancer therapy agent commonly used for treating said cancer.

In another aspect, the present invention relates to a Dbait molecule for use in a tumor delaying and/or preventing and/or reversing development of cancer resistant to a cancer therapy agent in a patient. The invention also relates to a Dbait molecule for use in extending the duration of response to a cancer therapy agent in a patient. The invention further relates to a Dbait molecule for use in extending the period of sensitivity of response to a cancer therapy agent in a patient. In some embodiments, the cancer therapy agent is chemotherapy. For instance, the cancer therapy agent can be selected from the group consisting of a platinum agent, an alkylating agent, a camptothecin, a nitrogen mustard, an antibiotic, an antimetabolite, and a vinca. Preferably, the cancer therapy agent is a platinum agent such as cisplatin, oxaliplatin and carboplatin. In other embodiments the cancer therapy agent is radiotherapy. In still other embodiments, the cancer therapy agent is a targeted therapy (e.g. a PARP inhibitor such as rucaparib (AG014699), olaparib (AZD2281), veliparib (ABT888), iniparib (BSI 201), niraparib (MK 4827), talazoparib (BMN673)). In a further embodiment, the cancer therapy agent is a Dbait molecule. Indeed, the Dbait molecule is suitable for increasing its auto sensitivity and delaying and/or preventing development of the resistance to itself.

The present invention also relates to a combination of a cancer therapy agent and a Dbait molecule for use in a method delaying and/or preventing development of cancer resistant to a cancer therapy agent. The present invention relates to method of treating a cancer in a patient by delaying and/or preventing development of cancer resistant to a cancer therapy agent, comprising administering to the patient an effective amount of (i) a cancer therapy agent, and (ii) a Dbait molecule, thereby delaying and/or preventing development of cancer resistant to the cancer therapy agent. In one embodiment, the cancer therapy agent and the Dbait molecule are administered concomitantly or simultaneously. In another embodiments, the Dbait molecule is administered after pretreatment with the cancer therapy agent. Optionally, the Dbait molecule is to be administered in combination therapy with the cancer therapy agent, preferably for at least two cycles of administration, more preferably for at least three or four cycles of administration.

In some embodiments, the cancer therapy agent is chemotherapy. For instance, the cancer therapy agent can be selected from the group consisting of a platinum agent, an alkylating agent, a camptothecin, a nitrogen mustard, an antibiotic, an antimetabolite, and a vinca. Preferably, the cancer therapy agent is a platinum agent such as cisplatin, oxaliplatin and carboplatin. In other embodiments the cancer therapy agent is radiotherapy. In still other embodiments, the cancer therapy agent is a targeted therapy (e.g. a PARP inhibitor such as rucaparib (AG014699), olaparib (AZD2281), veliparib (ABT888), iniparib (BSI 201), niraparib (MK 4827), talazoparib (BMN673)). In a further embodiment, the cancer therapy agent is a Dbait molecule. Indeed, the Dbait molecule is suitable for increasing its auto sensitivity and delaying and/or preventing development of the resistance to itself.

The present invention also relates to a combination of a cancer therapy agent and a Dbait molecule for use in a method of treating cancer in a patient by overcoming resistance of the cancer cells to the cancer therapy agent. The present invention relates to method of treating a cancer in a patient by overcoming resistance of the cancer cells to a cancer therapy agent, comprising administering to the patient an effective amount of (i) a cancer therapy agent, and (ii) a Dbait molecule. In one embodiment, the cancer therapy agent and the Dbait molecule are administered concomitantly or simultaneously. In another embodiments, the Dbait molecule is administered after pretreatment with the cancer therapy agent.

The present invention further relates to a combination of a cancer therapy agent and a Dbait molecule for use in a method of overcoming resistance of the cancer cells to the cancer therapy agent. The present invention relates to a method for overcoming drug-resistance of cancer cells in a patient, comprising administering to the patient an effective amount of (i) a cancer therapy agent, and (ii) a Dbait molecule. In one embodiment, the cancer therapy agent and the Dbait molecule are administered concomitantly or simultaneously. In another embodiment, the Dbait molecule is administered after pretreatment with the cancer therapy agent.

In some embodiments, the cancer therapy agent is chemotherapy. In other embodiments the cancer therapy agent is radiotherapy. In still other embodiments, the cancer therapy agent is a targeted therapy (e.g. a PARP inhibitor such as rucaparib (AG014699), olaparib (AZD2281), veliparib (ABT888), iniparib (BSI 201), niraparib (MK 4827), talazoparib (BMN673).

Cancer having resistance to a therapy as used herein includes a cancer which is not responsive and/or reduced ability of producing a significant response (e.g., partial response and/or complete response) to the therapy. Resistance may be acquired resistance which arises in the course of a treatment method. In some embodiments, the acquired drug resistance is transient and/or reversible drug tolerance. Transient and/or reversible drug resistance to a therapy includes wherein the drug resistance is capable of regaining sensitivity to the therapy after a break in the treatment method. In some embodiments, the acquired resistance is permanent resistance (including a genetic change conferring drug resistance).

Cancer having sensitivity to a therapy as used herein includes cancer which is responsive and/or capable of producing a significant response (e.g., partial response and/or complete response). Methods of determining of assessing acquisition of resistance and/or maintenance of sensitivity to a therapy are known in the art. Drug resistance and/or sensitivity may be determined by (a) exposing a reference cancer cell or cell population to a cancer therapy agent (e.g., targeted therapy, chemotherapy, and/or radiotherapy) in the presence and/or absence of Dbait molecule and/or (b) assaying, for example, for one or more of cancer cell growth, cell viability, level and/or percentage apoptosis and/or response.

Drug resistance and/or sensitivity may be measured over time and/or at various concentrations of cancer therapy agent (e.g., targeted therapy, chemotherapy, and/or radiotherapy) and/or amount of a Dbait molecule. Drug resistance and/or sensitivity further may be measured and/or compared to a reference cell line. In some embodiments, cell viability may be assayed by CyQuant Direct cell proliferation assay. In some embodiments, resistance may be indicated by a change in IC50, EC50 or decrease in tumor growth. In some embodiments, the change is greater than about any of 50%, 100%, and/or 200%. In addition, changes in acquisition of resistance and/or maintenance of sensitivity may be assessed in vivo for examples by assessing response, duration of response, and/or time to progression to a therapy, e.g., partial response and complete response. Changes in acquisition of resistance and/or maintenance of sensitivity may be based on changes in response, duration of response, and/or time to progression to a therapy in a population of individuals, e.g., number of partial responses and complete responses. The present invention relates to a Dbait molecule and a platinum agent for use in a method delaying and/or preventing and/or reversing development of cancer resistant to a platinum agent in a patient. The present invention also relates to a Dbait molecule and a platinum agent for use in a method delaying and/or preventing and/or reversing development of cancer resistant to a platinum agent in a patient, comprising sequentially, concomitantly or simultaneously administering to the patient (a) an effective amount of a Dbait molecule and (b) an effective amount of the platinum agent.

The present invention relates to a Dbait molecule and a platinum agent for use in a method of treating a patient with cancer who is resistant or has increased likelihood of developing resistance to a platinum agent. The present invention also relates to a Dbait molecule and a platinum agent for use in a method of treating a patient with cancer who is resistant or has increased likelihood of developing resistance to a platinum agent, comprising sequentially, concomitantly or simultaneously administering to the patient (a) an effective amount of a Dbait molecule and (b) an effective amount of the platinum agent.

The present invention relates to a Dbait molecule and a platinum agent for use in a method of extending the period of a platinum agent sensitivity in a patient with cancer. The present invention also relates to a Dbait molecule and a platinum agent for use in a method of extending the period of a platinum agent sensitivity in a patient with cancer comprising sequentially, concomitantly or simultaneously administering to the patient (a) an effective amount of a Dbait molecule and (b) an effective amount of the platinum agent.

The present invention relates to a Dbait molecule and a platinum agent for use in a method of extending the duration of response to a platinum agent in a patient. The present invention also relates to a Dbait molecule and a platinum agent for use in a method of extending the duration of response to a platinum agent in a patient comprising sequentially, concomitantly or simultaneously administering to the patient (a) an effective amount of a Dbait molecule and (b) an effective amount of the platinum agent.

The present invention relates to a Dbait molecule and a platinum agent for use in a method of treating cancer in a patient by overcoming resistance of the cancer cells to the platinum agent. The invention relates to a Dbait molecule and a platinum agent for use in a method of treating cancer in a patient by overcoming resistance of the cancer cells to the platinum agent in a patient comprising sequentially, concomitantly or simultaneously administering to the patient (a) an effective amount of a Dbait molecule and (b) an effective amount of the platinum agent.

The present invention relates to a Dbait molecule and a platinum agent for use in a method of overcoming resistance of the cancer cells to the platinum agent. The present invention also relates to a Dbait molecule and a platinum agent for use in a method of overcoming resistance of the cancer cells to the platinum agent in a patient comprising sequentially, concomitantly or simultaneously administering to the patient (a) an effective amount of a Dbait molecule and (b) an effective amount of the platinum agent.

In some embodiments, the platinum agent useful in the methods and uses described above are selected from the group consisting of cisplatin, oxaliplatin and carboplatin and the Dbait molecule is AsiDNA.

The present invention relates to a Dbait molecule and a PARP inhibitor for use in a method delaying and/or preventing and/or reversing development of cancer resistant to a PARP inhibitor in a patient. The present invention also relates to a Dbait molecule and a PARP inhibitor for use in a method delaying and/or preventing and/or reversing development of cancer resistant to a PARP inhibitor in a patient, comprising sequentially, concomitantly or simultaneously administering to the individual (a) an effective amount of a Dbait molecule and (b) an effective amount of the PARP inhibitor.

The present invention relates to a Dbait molecule and a platinum agent for use in a method of treating a patient with cancer who is resistant or has increased likelihood of developing resistance to a PARP inhibitor. The present invention relates to a Dbait molecule and a platinum agent for use in a method of treating a patient with cancer who is resistant or has increased likelihood of developing resistance to a PARP inhibitor, comprising sequentially, concomitantly or simultaneously administering to the patient (a) an effective amount of a Dbait molecule and (b) an effective amount of the PARP inhibitor.

The present invention relates to a Dbait molecule and a PARP inhibitor for use in a method of extending the period of a PARP inhibitor sensitivity in a patient with cancer. The present invention relates to a Dbait molecule and a PARP inhibitor for use in a method of extending the period of a PARP inhibitor sensitivity in a patient with cancer comprising sequentially, concomitantly or simultaneously administering to the patient (a) an effective amount of a Dbait molecule and (b) an effective amount of the PARP inhibitor.

The present invention relates to a Dbait molecule and a PARP inhibitor for use in a method of extending the duration of response to a PARP inhibitor. The present invention relates to a Dbait molecule and a PARP inhibitor for use in a method of extending the duration of response to a PARP inhibitor in a patient comprising sequentially, concomitantly or simultaneously administering to the patient (a) an effective amount of a Dbait molecule and (b) an effective amount of the PARP inhibitor.

The present invention relates to a Dbait molecule and PARP inhibitor for use in a method of treating cancer in a patient by overcoming resistance of the cancer cells to the PARP inhibitor. The present invention also relates to a Dbait molecule and PARP inhibitor for use in a method of treating cancer in a patient by overcoming resistance of the cancer cells to the PARP inhibitor comprising sequentially, concomitantly or simultaneously administering to the patient (a) an effective amount of a Dbait molecule and (b) an effective amount of the PARP inhibitor.

The present invention relates to a Dbait molecule and a PARP inhibitor for use in a method of overcoming resistance of the cancer cells to the PARP inhibitor. The present invention also relates to a Dbait molecule and a PARP inhibitor for use in a method of overcoming resistance of the cancer cells to the PARP inhibitor comprising sequentially, concomitantly or simultaneously administering to the patient (a) an effective amount of a Dbait molecule and (b) an effective amount of the PARP inhibitor.

In some embodiments, the PARP inhibitor useful in the methods and uses described above are selected from the group consisting of rucaparib, olaparib, veliparib, iniparib, niraparib and talazoparib and the Dbait molecule is AsiDNA.

Dbait Molecules:

The term “Dbait molecule” also known as signal interfering DNA (siDNA) as used herein, refers to a nucleic acid molecule, preferably a hairpin nucleic acid molecule, designed to counteract DNA repair. A Dbait molecule has at least one free end and a DNA double stranded portion of 6-200 bp with less than 60% sequence identity to any gene in a human genome.

Preferably, the Dbait molecules for use in the present invention, conjugated or not, can be described by the following formulae:

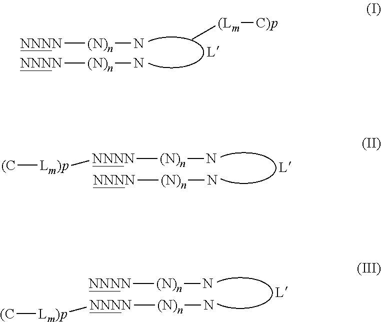

wherein N is a deoxynucleotide, n is an integer from 1 to 195, the underlined N refers to a nucleotide having or not a modified phosphodiester backbone, L′ is a linker, C is a molecule facilitating endocytosis preferably selected from a lipophilic molecule and a ligand which targets cell receptor enabling receptor mediated endocytosis, L is a linker, m and p, independently, are an integer being 0 or 1.

In preferred embodiments, the Dbait molecules of formulae (I), (II), or (III) have one or several of the following features:
N is a deoxynucleotide, preferably selected from the group consisting of A (adenine), C (cytosine), T (thymine) and G (guanine) and selected so as to avoid occurrence of a CpG dinucleotide and to have less than 80% or 70%, even less than 60% or 50% sequence identity to any gene in a human genome; and/or,
n is an integer from 1 to 195, preferably from 3 to 195, optionally from 1 to 95, from 2 to 95, from 3 to 95, from 5 to 95, from 15 to 195, from 19-95, from 21 to 95, from 27 to 95, from 1 to 45, from 2 to 35, from 3 to 35, from 5 to 35, from 15 to 45, from 19 to 45, from 21 to 45, or from 27 to 45. In a particularly preferred embodiment, n is 27; and/or,
the underlined N refers to a nucleotide having or not a phosphorothioate or methylphosphonate backbone, more preferably a phosphorothioate backbone; preferably, the underlined N refers to a nucleotide having a modified phosphodiester backbone; and/or,
the linker L′ is selected from the group consisting of hexaethyleneglycol, tetradeoxythymidylate (T4), 1,19-bis (phospho)-8-hydraza-2-hydroxy-4-oxa-9-oxo-nonadecane; and 2,19-bis (phosphor)-8-hydraza-1-hydroxy-4-oxa-9-oxo-nonadecane and/or,
m is 1 and L is a carboxamido polyethylene glycol, more preferably carboxamido triethylene or tetraethylene glycol; and/or,
C is selected from the group consisting of a cholesterol, single or double chain fatty acids such as octadecyl, oleic acid, dioleoyl or stearic acid, or ligand (including peptide, protein, aptamer) which targets cell receptor such as folic acid, tocopherol, sugar such as galactose and mannose and their oligosaccharide, peptide such as RGD and bombesin, and protein such transferring and integrin, preferably is a cholesterol or a tocopherol, still more preferably a cholesterol.

Preferably, C-Lm is a triethyleneglycol linker (10-O-[1-propyl-3-N-carbamoylcholesteryl]-triethyleneglycol radical. Alternatively, C-Lm is a tetraethyleneglycol linker (10-O-[1-propyl-3-N-carbamoylcholesteryl]-tetraethyleneglycol radical.

In a preferred embodiment, the Dbait molecule has the following formula:

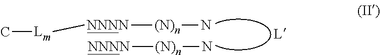

with the same definition than formulae (I), (II), and (III) for N, N, n, L, L′, C and m. In a particular embodiment, the Dbait molecules are those extensively described in PCT patent applications WO2005/040378, WO2008/034866, WO2008/084087 and WO2011/161075, the disclosure of which is incorporated herein by reference.

Dbait molecules may be defined by a number of characteristics necessary for their therapeutic activity, such as their minimal length, the presence of at least one free end, and the presence of a double stranded portion, preferably a DNA double stranded portion. As will be discussed below, it is important to note that the precise nucleotide sequence of Dbait molecules does not impact on their activity. Furthermore, Dbait molecules may contain a modified and/or non-natural backbone.

Preferably, Dbait molecules are of non-human origin (i.e., their nucleotide sequence and/or conformation (e.g., hairpin) does not exist as such in a human cell), most preferably of synthetic origin. As the sequence of the Dbait molecules plays little, if any, role, Dbait molecules have preferably no significant degree of sequence homology or identity to known genes, promoters, enhancers, 5′- or 3′-upstream sequences, exons, introns, and the like. In other words, Dbait molecules have less than 80% or 70%, even less than 60% or 50% sequence identity to any gene in a human genome. Methods of determining sequence identity are well known in the art and include, e.g., Blast. Dbait molecules do not hybridize, under stringent conditions, with human genomic DNA. Typical stringent conditions are such that they allow the discrimination of fully complementary nucleic acids from partially complementary nucleic acids.

In addition, the sequence of the Dbait molecules is preferably devoid of CpG in order to avoid the well-known toll-like receptor-mediated immunological reactions.

The length of Dbait molecules may be variable, as long as it is sufficient to allow appropriate binding of Ku protein complex comprising Ku and DNA-PKcs proteins. It has been showed that the length of Dbait molecules must be greater than 20 bp, preferably about 32 bp, to ensure binding to such a Ku complex and allowing DNA-PKcs activation. Preferably, Dbait molecules comprise between 20-200 bp, more preferably 24-100 bp, still more preferably 26-100, and most preferably between 24-200, 25-200, 26-200, 27-200, 28-200, 30-200, 32-200, 24-100, 25-100, 26-100, 27-100, 28-100, 30-100, 32-200 or 32-100 bp. For instance, Dbait molecules comprise between 24-160, 26-150, 28-140, 28-200, 30-120, 32-200 or 32-100 bp. By “bp” is intended that the molecule comprise a double stranded portion of the indicated length.

In a particular embodiment, the Dbait molecules having a double stranded portion of at least 32 pb, or of about 32 bp, comprise the same nucleotide sequence than Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5). Optionally, the Dbait molecules have the same nucleotide composition than Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5) but their nucleotide sequence is different. Then, the Dbait molecules comprise one strand of the double stranded portion with 3 A, 6 C, 12 G and 11 T. Preferably, the sequence of the Dbait molecules does not contain any CpG dinucleotide.

Alternatively, the double stranded portion comprises at least 16, 18, 20, 22, 24, 26, 28, 30 or 32 consecutive nucleotides of Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5). In a more particular embodiment, the double stranded portion consists in 20, 22, 24, 26, 28, 30 or 32 consecutive nucleotides of Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5).

The Dbait molecules as disclosed herein must have at least one free end, as a mimic of double strand breaks (DSB). Said free end may be either a free blunt end or a 5′-/3′-protruding end. The “free end” refers herein to a nucleic acid molecule, in particular a double-stranded nucleic acid portion, having both a 5′ end and a 3′ end or having either a 3′ end or a 5′ end. Optionally, one of the 5′ and 3′ end can be used to conjugate the nucleic acid molecule or can be linked to a blocking group, for instance a or 3′-3′nucleotide linkage.

In a particular embodiment, they contain only one free end. Preferably, Dbait molecules are made of hairpin nucleic acids with a double-stranded DNA stem and a loop. The loop can be a nucleic acid, or other chemical groups known by skilled person or a mixture thereof. A nucleotide linker may include from 2 to 10 nucleotides, preferably, 3, 4 or 5 nucleotides. Non-nucleotide linkers non exhaustively include abasic nucleotide, polyether, polyamine, polyamide, peptide, carbohydrate, lipid, polyhydrocarbon, or other polymeric compounds (e. g. oligoethylene glycols such as those having between 2 and 10 ethylene glycol units, preferably 3, 4, 5, 6, 7 or 8 ethylene glycol units). A preferred linker is selected from the group consisting of hexaethyleneglycol, tetradeoxythymidylate (T4) and other linkers such as 1,19-bis (phospho)-8-hydraza-2-hydroxy-4-oxa-9-oxo-nonadecane and 2,19-bis (phosphor)-8-hydraza-1-hydroxy-4-oxa-9-oxo-nonadecane. Accordingly, in a particular embodiment, the Dbait molecules can be a hairpin molecule having a double stranded portion or stem comprising at least 16, 18, 20, 22, 24, 26, 28, 30 or 32 consecutive nucleotides of Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5) and a loop being a hexaethyleneglycol linker, a tetradeoxythymidylate linker (T4) 1,19-bis(phospho)-8-hydraza-2-hydroxy-4-oxa-9-oxo-nonadecane or 2,19-bis(phosphor)-8-hydraza-1-hydroxy-4-oxa-9-oxo-nonadecane. In a more particular embodiment, those Dbait molecules can have a double stranded portion consisting in 20, 22, 24, 26, 28, 30 or 32 consecutive nucleotides of Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5).

Dbait molecules preferably comprise a 2′-deoxynucleotide backbone, and optionally comprise one or several (2, 3, 4, 5 or 6) modified nucleotides and/or nucleobases other than adenine, cytosine, guanine and thymine. Accordingly, the Dbait molecules are essentially a DNA structure. In particular, the double-strand portion or stem of the Dbait molecules is made of deoxyribonucleotides.

Preferred Dbait molecules comprise one or several chemically modified nucleotide(s) or group(s) at the end of one or of each strand, in particular in order to protect them from degradation. In a particular preferred embodiment, the free end(s) of the Dbait molecules is(are) protected by one, two or three modified phosphodiester backbones at the end of one or of each strand. Preferred chemical groups, in particular the modified phosphodiester backbone, comprise phosphorothioates. Alternatively, preferred Dbait have 3′-3′ nucleotide linkage, or nucleotides with methylphosphonate backbone. Other modified backbones are well known in the art and comprise phosphoramidates, morpholino nucleic acid, 2′-0,4′-C methylene/ethylene bridged locked nucleic acid, peptide nucleic acid (PNA), and short chain alkyl, or cycloalkyl intersugar linkages or short chain heteroatomic or heterocyclic intrasugar linkages of variable length, or any modified nucleotides known by skilled person. In a first preferred embodiment, the Dbait molecules have the free end(s) protected by one, two or three modified phosphodiester backbones at the end of one or of each strand, more preferably by three modified phosphodiester backbones (in particular phosphorothioate or methylphosphonate) at least at the 3′ end, but still more preferably at both 5′ and 3′ ends.

In a most preferred embodiment, the Dbait molecule is a hairpin nucleic acid molecule comprising a DNA double-stranded portion or stem of 32 bp (e.g., with a sequence selected from the group consisting of SEQ ID Nos 1-5, in particular SEQ ID No 4) and a loop linking the two strands of the DNA double-stranded portion or stem comprising or consisting of a linker selected from the group consisting of hexaethyleneglycol, tetradeoxythymidylate (T4) and 1,19-bis (phospho)-8-hydraza-2-hydroxy-4-oxa-9-oxo-nonadecane and 2,19-bis(phosphor)-8-hydraza-1-hydroxy-4-oxa-9-oxo-nonadecane, the free ends of the DNA double-stranded portion or stem (i.e. at the opposite of the loop) having three modified phosphodiester backbones (in particular phosphorothioate internucleotidic links).

Said nucleic acid molecules are made by chemical synthesis, semi-biosynthesis or biosynthesis, any method of amplification, followed by any extraction and preparation methods and any chemical modification. Linkers are provided so as to be incorporable by standard nucleic acid chemical synthesis. More preferably, nucleic acid molecules are manufactured by specially designed convergent synthesis: two complementary strands are prepared by standard nucleic acid chemical synthesis with the incorporation of appropriate linker precursor, after their purification, they are covalently coupled together.

Optionally, the nucleic acid molecules may be conjugated to molecules facilitating endocytosis or cellular uptake.

In particular, the molecules facilitating endocytosis or cellular uptake may be lipophilic molecules such as cholesterol, single or double chain fatty acids, or ligands which target cell receptor enabling receptor mediated endocytosis, such as folic acid and folate derivatives or transferrin (Goldstein et al. Ann. Rev. Cell Biol. 1985 1:1-39; Leamon & Lowe, Proc Natl Acad Sci USA. 1991, 88: 5572-5576.). The molecule may also be tocopherol, sugar such as galactose and mannose and their oligosaccharide, peptide such as RGD and bombesin and protein such as integrin. Fatty acids may be saturated or unsaturated and be in C4-C28, preferably in C14-C22, still more preferably being in C18 such as oleic acid or stearic acid. In particular, fatty acids may be octadecyl or dioleoyl. Fatty acids may be found as double chain form linked with in appropriate linker such as a glycerol, a phosphatidylcholine or ethanolamine and the like or linked together by the linkers used to attach on the Dbait molecule. As used herein, the term “folate” is meant to refer to folate and folate derivatives, including pteroic acid derivatives and analogs. The analogs and derivatives of folic acid suitable for use in the present invention include, but are not limited to, antifolates, dihydrofolates, tetrahydrofolates, folinic acid, pteropolyglutamic acid, 1-deza, 3-deaza, 5-deaza, 8-deaza, 10-deaza, 1,5-deaza, 5,10 dideaza, 8,10-dideaza, and 5,8-dideaza folates, antifolates, and pteroic acid derivatives. Additional folate analogs are described in US2004/242582. Accordingly, the molecule facilitating endocytosis may be selected from the group consisting of single or double chain fatty acids, folates and cholesterol. More preferably, the molecule facilitating endocytosis is selected from the group consisting of dioleoyl, octadecyl, folic acid, and cholesterol. In a most preferred embodiment, the nucleic acid molecule is conjugated to a cholesterol.

The Dbait molecules facilitating endocytosis may conjugated to molecules facilitating endocytosis, preferably through a linker. Any linker known in the art may be used to attach the molecule facilitating endocytosis to Dbait molecules For instance, WO09/126933 provides a broad review of convenient linkers pages 38-45. The linker can be non-exhaustively, aliphatic chain, polyether, polyamine, polyamide, peptide, carbohydrate, lipid, polyhydrocarbon, or other polymeric compounds (e. g. oligoethylene glycols such as those having between 2 and 10 ethylene glycol units, preferably 3, 4, 5, 6, 7 or 8 ethylene glycol units, still more preferably 3 ethylene glycol units), as well as incorporating any bonds that may be break down by chemical or enzymatical way, such as a disulfide linkage, a protected disulfide linkage, an acid labile linkage (e.g., hydrazone linkage), an ester linkage, an ortho ester linkage, a phosphonamide linkage, a biocleavable peptide linkage, an azo linkage or an aldehyde linkage. Such cleavable linkers are detailed in WO2007/040469 pages 12-14, in WO2008/022309 pages 22-28.

In a particular embodiment, the nucleic acid molecule can be linked to one molecule facilitating endocytosis. Alternatively, several molecules facilitating endocytosis (e.g., two, three or four) can be attached to one nucleic acid molecule.

In a specific embodiment, the linker between the molecule facilitating endocytosis, in particular cholesterol, and nucleic acid molecule is CO—NH—(CH₂—CH₂—O)ₙ, wherein n is an integer from 1 to 10, preferably n being selected from the group consisting of 3, 4, 5 and 6. In a very particular embodiment, the linker is CO—NH—(CH₂—CH₂—O)₄ (carboxamido tetraethylene glycol) or CO—NH—(CH₂—CH₂—O)₃ (carboxamido triethylene glycol). The linker can be linked to nucleic acid molecules at any convenient position which does not modify the activity of the nucleic acid molecules. In particular, the linker can be linked at the 5′ end. Therefore, in a preferred embodiment, the contemplated conjugated Dbait molecule is a Dbait molecule having a hairpin structure and being conjugated to the molecule facilitating endocytosis, preferably through a linker, at its 5′ end.

In another specific embodiment, the linker between the molecule facilitating endocytosis, in particular cholesterol, and nucleic acid molecule is dialkyl-disulfide {e.g., (CH₂)ᵣ—S—S—(CH₂)ₛ with r and s being integer from 1 to 10, preferably from 3 to 8, for instance 6}.

In a most preferred embodiment, the conjugated Dbait molecule is a hairpin nucleic acid molecule comprising a DNA double-stranded portion or stem of 32 bp and a loop linking the two strands of the DNA double-stranded portion or stem comprising or consisting of a linker selected from the group consisting of hexaethyleneglycol, tetradeoxythymidylate (T4), 1,19-bis (phospho)-8-hydraza-2-hydroxy-4-oxa-9-oxo-nonadecane and 2,19-bis (phosphor)-8-hydraza-1-hydroxy-4-oxa-9-oxo-nonadecane, the free ends of the DNA double-stranded portion or stem (i.e. at the opposite of the loop) having three modified phosphodiester backbones (in particular phosphorothioate internucleotidic links) and said Dbait molecule being conjugated to a cholesterol at its 5′ end, preferably through a linker (e.g. carboxamido oligoethylene glycol, preferably carboxamido triethylene or tetraethylene glycol).

In a particular embodiment, the Dbait molecules can be conjugated Dbait molecules such as those extensively described in PCT patent application WO2011/161075, the disclosure of which is incorporated herein by reference.

In a preferred embodiment, NNNN—(N)ₙ—N comprises at least 6, 8, 10, 12, 14, 16, 18, 20, 22, 24, 26, 28, 30 or 32 consecutive nucleotides of Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5) or consists in 20, 22, 24, 26, 28, 30 or 32 consecutive nucleotides of Dbait32, Dbait32Ha, Dbait32Hb, Dbait32Hc or Dbait32Hd. In a particular embodiment, NNNN—(N)ₙ—N comprises or consists in Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5), more preferably Dbait32Hc (SEQ ID NO: 4).

According, the conjugated Dbait molecules may be selected from the group consisting of:

with NNNN—(N)ₙ—N being SEQ ID NO: 1;

with NNNN—(N)ₙ—N being SEQ ID NO: 2;

with NNNN—(N)ₙ—N being SEQ ID NO: 3;

with NNNN—(N)ₙ—N being SEQ ID NO: 4; or

with NNNN—(N)ₙ—N being SEQ ID NO: 5

In one preferred embodiment, the Dbait molecule has the following formula:

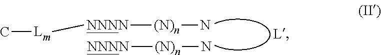

wherein
NNNN—(N)ₙ—N comprises 28, 30 or 32 nucleotides, preferably 32 nucleotides and/or
the underlined nucleotide refers to a nucleotide having or not a phosphorothioate or methylphosphonate backbone, more preferably a phosphorothioate backbone; preferably, the underlined nucleotide refers to a nucleotide having a phosphorothioate or methylphosphonate backbone, more preferably a phosphorothioate backbone and/or,
the linker L′ is selected from the group consisting of hexaethyleneglycol, tetradeoxythymidylate (T4), 1,19-bis (phospho)-8-hydraza-2-hydroxy-4-oxa-9-oxo-nonadecane or 2,19-bis(phosphor)-8-hydraza-1-hydroxy-4-oxa-9-oxo-nonadecane; and/or,
m is 1 and L is a carboxamido polyethylene glycol, more preferably carboxamido triethylene or tetraethylene glycol; and/or,
p is 1; and/or,
C is selected from the group consisting of a cholesterol, single or double chain fatty acids such as octadecyl, oleic acid, dioleoyl or stearic acid, or ligand (including peptide, protein, aptamer) which targets cell receptor such as folic acid, tocopherol, sugar such as galactose and mannose and their oligosaccharide, peptide such as RGD and bombesin, and protein such transferring and integrin, preferably is a cholesterol.

In a very specific embodiment, the Dbait molecule (also referred herein as AsiDNA) has the following formula:

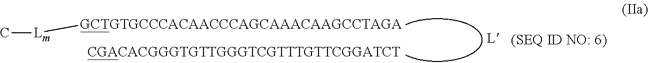

wherein C-Lₘ is the tetraethyleneglycol linker (10-O-[1-propyl-3-N-carbamoylcholesteryl]-tetraethyleneglycol radical, and L′ is 1,19-bis(pho spho)-8-hydraza-2-hydroxy-4-oxa-9-oxo-nonadecane; also represented by the following formula:

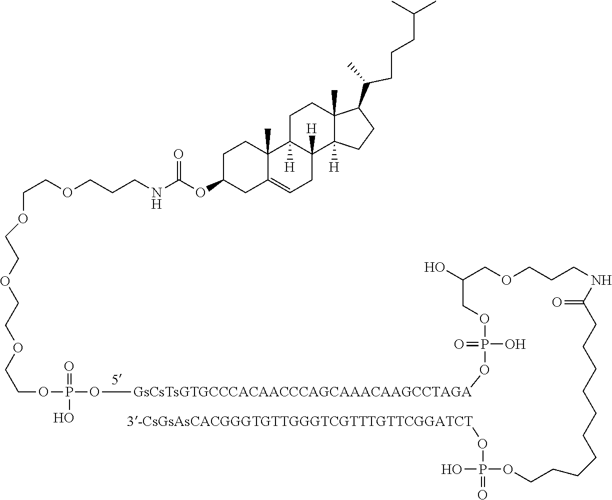

Cancers or Tumors to be Treated:

The terms “cancer”, “cancerous”, or “malignant” refer to or describe the physiological condition in mammals that is typically characterized by unregulated cell growth. Examples of cancer include, for example, leukemia, lymphoma, blastoma, carcinoma and sarcoma.

Various cancers are also encompassed by the scope of the invention, including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testis, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma (including cutaneous or peripheral T-cell lymphoma), Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyosarcoma, and osteosarcoma; other tumors including melanoma, xenoderma pigmentosum, keratoactanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatocarcinoma, breast cancer, colon carcinoma, and head and neck cancer, retinoblastoma, gastric cancer, germ cell tumor, bone cancer, bone tumors, adult malignant fibrous histiocytoma of bone; childhood malignant fibrous histiocytoma of bone, sarcoma, pediatric sarcoma; myelodysplastic syndromes; neuroblastoma; testicular germ cell tumor, intraocular melanoma, myelodysplastic syndromes; myelodysplastic/myeloproliferative diseases, synovial sarcoma. In a preferred embodiment of the present invention, the cancer is a solid tumor. For instance, the cancer may be sarcoma and osteosarcoma such as Kaposi sarcome, AIDS-related Kaposi sarcoma, melanoma, in particular uveal melanoma, and cancers of the head and neck, kidney, ovary, pancreas, prostate, thyroid, lung, esophagus, breast in particular triple negative breast cancer (TNBC), bladder, colorectum, liver and biliary tract, uterine, appendix, and cervix, testicular cancer, gastrointestinal cancers and endometrial and peritoneal cancers.

### EXAMPLES

### Example 1: AsiDNA-Sensitizes Tumor Cells to AsiDNA Treatment

Materials and Methods

Cell Culture

Triple negative breast cancer cell line MDA-MB-231 were purchased from ATCC and grown according to the supplier's instructions. Briefly, MDA-MB-231 cells are grown in L15 Leibovitz medium supplemented with 10% fetal bovine serum (FBS) and maintained in a humidified atmosphere at 37° C. and 0% CO₂.

AsiDNA Treatment and Measurement of Cellular Survival

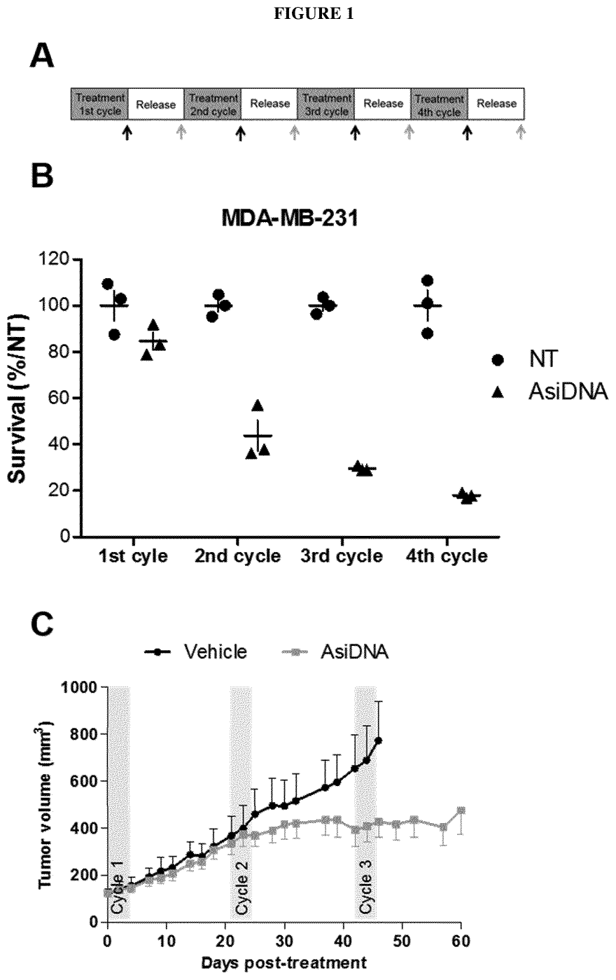

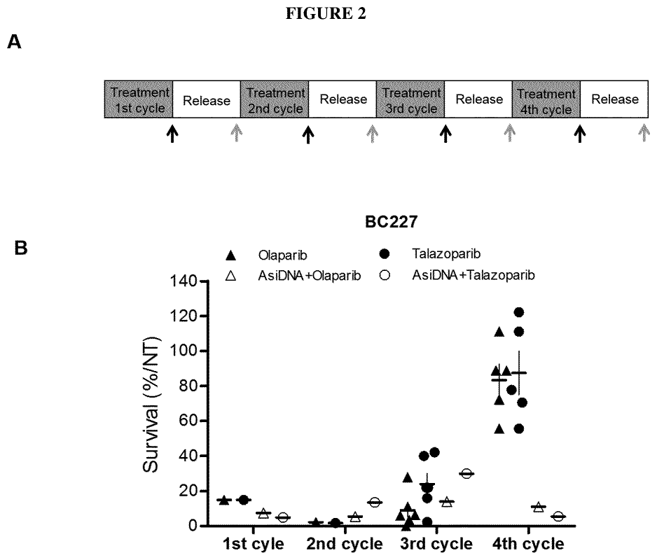

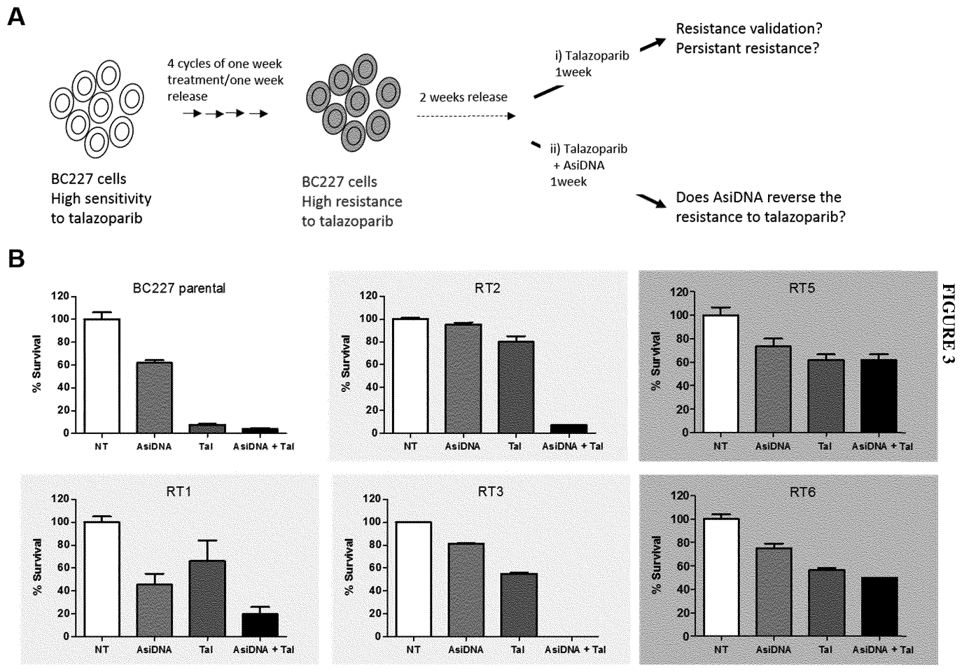

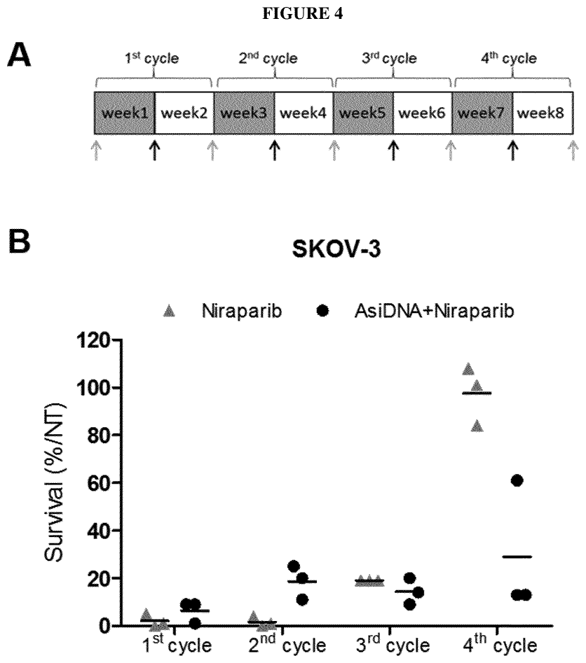

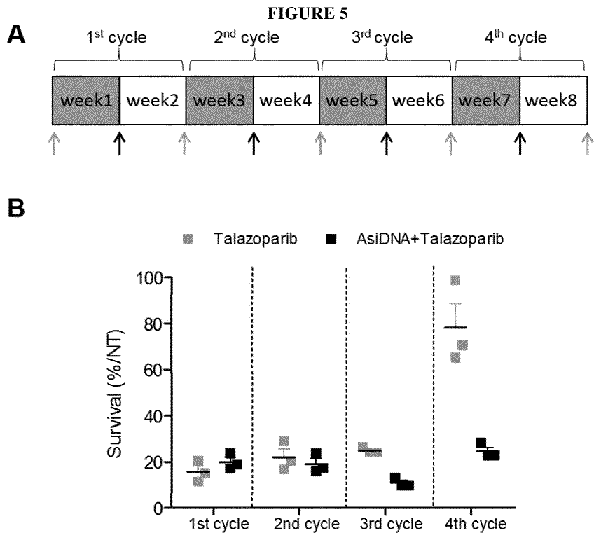

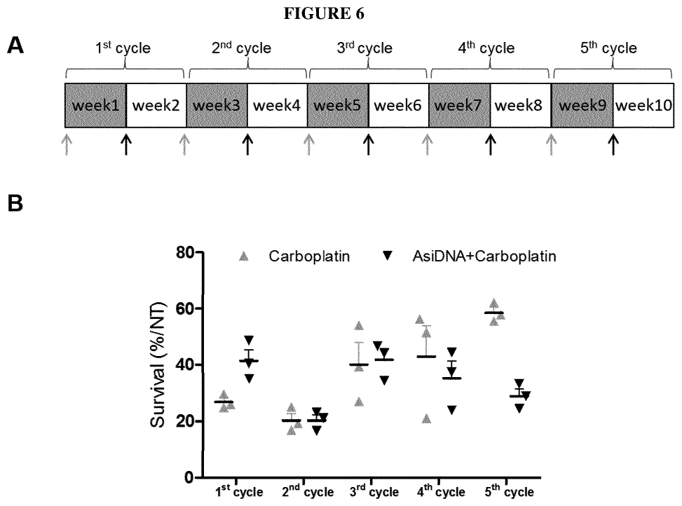

Cells were seeded in 6-well culture plates at appropriate densities and incubated 24 h at 37° C. before AsiDNA addition. Cells were harvested on day 7 after treatment, stained with 0.4% trypan blue (Sigma Aldrich, Saint-Louis, USA) and counted under microscope using Kova slides. Cell survival was calculated as the ratio of living treated cells/living not-treated cells. Cell death was calculated as the number of dead cells out of the total number of counted cells. Cells are then washed to remove AsiDNA, and again seeded in 6-well culture plates for recovery during 6 days. A second cycle of treatment/recovery was then started. Four cycles were performed. (FIG. 1A).

In Vivo Experiments

MDA-MB-231 cell-derived-xenografts (CDXs) were obtained by injecting 5.10⁶ cells into the mammary fat pad of six to eight-week-old adult female nude NMRI-nu Rj:NMRI-Foxn1^{nu}/Foxn1^{nu} mice (Janvier). The animals were housed at least one week before tumor engraftment under controlled conditions of light and dark (12 h/12 h), relative humidity (55%), and temperature (21° C.). Mice were randomized into different treatment groups of 10-15 animals when engrafted tumors reached 80-250 mm³. AsiDNA was injected systemically (intraperitoneal administration). Tumor growth was evaluated three times a week using a caliper and tumor volume was calculated using the following formula: (length×width)/2. Mice were followed for up to three months, and ethically sacrificed when the tumor volume reached 1,500 mm³. The Local Animal Experimentation Ethics Committee approved all experiments. The authorization to perform animal studies (#01593.02) was delivered by the French Ministère de l'Education Nationale, de l'Enseignement Supérieur et de la Recherche.

Results

It has been shown in several reports that the more accurate protocol to generate chemotherapy or Targeted therapies resistance is to submit tumor cells to repeated cycles of treatment, to favor the selection of resistant clones (Galluzzi L, et al., Cell Rep. 2012 Aug. 30; 2(2):257-69; Michels J, et al., Cancer Res. 2013 Apr. 1; 73(7):2271-80). We used this protocol to test the effect of AsiDNA on the survival of triple negative breast cancer cell line MDA-MB-231.

Protocol and results are shown in FIG. 1.

We performed repeated cycles of AsiDNA treatment (504) in three independent populations of MDA-MB-231 cells. Four cycles of AsiDNA treatment in a schedule of “one week treatment-one week recovery” (FIG. 1A) were performed to repopulate the living cell pool between each treatment cycle. No resistant clones to AsiDNA have been isolated during repeated treatment. In contrast, we observed a therapy-induced increase of sensitivity to AsiDNA following repeated cycles (FIG. 1B). In fact, MDA-MB-231 cells display low sensitivity to AsiDNA at a dose of 504 (85% survival compared to non-treated populations). After the second cycle of treatment 45% of cells survive instead of 85% after the first cycle. After the fourth cycle only 20% of cells survive demonstrating that repeated treatments with AsiDNA increase cancer cells sensitivity rather than resistance emergence (FIG. 1B).

We validated in vivo the increase of sensitivity to AsiDNA upon cyclic treatments in MDA-MB-231 cell-derived xenografts (FIG. 1C). The tumors did not respond to the first cycle of treatment but stopped growing at the second treatment cycle, suggesting that tumors might also develop sensitivity with repeated treatment. These results are consistent with in vitro data, which showed a large increase in sensitivity to AsiDNA starting from the second cycle of AsiDNA treatment (FIG. 1B).

### Example 2: AsiDNA Abrogates the Emergence of Resistance to PARP Inhibitors in Breast Cancer

Materials and Methods

Cell Culture

Triple negative breast cancer cell line BC227 (BRCA2^{-/-}; a patient-derived cell line, from Curie institute) are grown according to the supplier's instructions. BC227 cell line are grown in DMEM medium supplemented with 10% FBS and 10 μg/ml insulin and maintained in a humidified atmosphere at 37° C. and 5% CO₂.

Drug Treatment and Measurement of Cellular Survival

For repeated cycles of treatment protocol, the drug cytotoxicity was measured by relative survival and cell death quantification. Cells were seeded in 6-well culture plates at appropriate densities and incubated 24 hat 37° C. before drug addition (olaparib 10 μM or talazoparib 0.1 μM with or without AsiDNA 2.504). Cells were harvested on day 7 after treatment, stained with 0.4% trypan blue (Sigma Aldrich, Saint-Louis, USA) and counted under microscope using Kova slides. Cell survival was calculated as the ratio of living treated cells/living not-treated cells. Cell death was calculated as the number of dead cells out of the total number of counted cells. Cells are then washed to remove drugs, and again seeded in 6-well culture plates for recovery during 6 days. A second cycle of treatment/recovery was then started. Four cycles were performed (FIG. 2A).

Results

Conventional anticancer treatments, and more recently targeted therapies, have improved the control of tumors, but side effects that limit dose escalation and the onset of resistance during treatment, are the prime causes of therapy failure. Under the selective pressure of anti-cancer treatments, resistant populations of cancer cells invariably evolve giving rise to “resistant clones” that have adapted to the new environment induced by the treatment.

In this context, we evaluated the effect of AsiDNA co-treatment on acquired resistance to PARP inhibitors onset.

Development of poly (adenosine disphosphate [ADP]-ribose) polymerase inhibitors (PARPi) to treat patients with inefficient HR repair have been the first example of exploiting DNA repair defects to treat cancer. However, mechanisms of resistance to this only FDA-approved family of DNA repair inhibitors have been described in preclinical models and in clinic (Chiarugi A. Trends Pharmacol Sci. 2012 January; 33(1):42-8). BC227 cells (BRCA2^{-/-}; HR deficient), initially very sensitive to PARPi, have been treated with repeated cycles of olaparib (10 μM) or talazoparib (100 nM) (High doses corresponding to the IC90) with or without AsiDNA (low dose—2.5 μM). Five independent populations of each treatment have been grown and maintained during four cycles of treatment (FIG. 2A). Clear resistance has been observed to olaparib and talazoparib in all the cell independent populations (FIG. 2B). Interestingly, cell populations treated with both PARPi and AsiDNA, remains very sensitive to the drugs demonstrating that AsiDNA, at a low dose, abrogated the emergence of acquired resistance to both PARPi. These results strongly suggest that AsiDNA could be used in combination with PARP inhibitors for breast cancer therapies to delay or abrogate PARP inhibitor acquired resistance onset.

### Example 3: AsiDNA Reverses the Acquired Resistance to Talazoparib in Breast Cancer

Materials and Methods

Cell Culture

Triple negative breast cancer cell line BC227 (BRCA2^{-/-}; a patient-derived cell line, from Curie institute) are grown according to the supplier's instructions. BC227 cell line are grown in DMEM medium supplemented with 10% FBS and 10 μg/ml insulin and maintained in a humidified atmosphere at 37° C. and 5% CO₂.

Drug Treatment and Measurement of Cellular Survival

For repeated cycles of treatment protocol, talazoparib (100 nM) cytotoxicity was measured by relative survival and cell death quantification. Cells were seeded in 6-well culture plates at appropriate densities and incubated 24 h at 37° C. before drug addition. Cells were harvested on day 7 after treatment, stained with 0.4% trypan blue (Sigma Aldrich, Saint-Louis, USA) and counted under microscope using Kova slides. Cell survival was calculated as the ratio of living treated cells/living not-treated cells. Cell death was calculated as the number of dead cells out of the total number of counted cells. Cells are then washed to remove talazoparib, and again seeded in 6-well culture plates for recovery during 6 days. A second cycle of treatment/recovery was then started. Four cycles were performed and BC227 resistant populations to talazoparib were subcultered for two weeks, then treated with either talazoparib (100 nM) to validate the acquired resistance or with talazoparib+AsiDNA to test if AsiDNA (504) could reverse talazoparib resistance (FIG. 3A).

Results

Much efforts are made to find the appropriate protocol of treatment to reverse acquired resistance to targeted therapies. In this context, the inventors tested if adding AsiDNA could reverse the acquired resistance to the PARP inhibitor talazoparib.

More particularly, the results are shown in FIG. 3.

Four cycles of one week treatment/one week release were sufficient to generate resistance to the PARP inhibitor talazoparib in the initially very sensitive parental BC227 cell line. The five independent resistant populations to talazoparib have been subcultered for two weeks without treatment (populations RT1, RT2, RT3, RT5 and RT6), and then treated either with talazoparib (100 nM) to validate the persistence of the resistance, or with the combined treatment talazoparib+AsiDNA to check if AsiDNA could reverse talazoparib resistance (FIG. 3A). Resistance to talazoparib (100 nM) was maintained in the five independent populations (survival between 50 and 90%) compared to BC227 control cells (10% survival) (FIG. 3B). Interestingly, adding AsiDNA to talazoparib during one week reversed this resistance (survival between 0 and 20%) in three resistant populations out of five (RT1, RT2 and RT3). Other cycles of treatment are ongoing to see if more cycles of combined treatment are needed to reverse talazoparib resistance in RT5 and RT6 populations.

### Example 4: AsiDNA Abrogates the Emergence of Resistance to Niraparib in Ovarian Cancer

Materials and Methods

Cell Culture

The ovarian cancer cell line SKOV-3 was grown according to the supplier's instructions, in McCoy's 5a medium supplemented with 10% FBS and maintained in a humidified atmosphere at 37° C. and 5% CO₂.

Drug Treatment and Measurement of Cellular Survival

For repeated cycles of treatment protocol, the drug cytotoxicity was measured by relative survival and cell death quantification. Cells were seeded in 6-well culture plates at appropriate densities and incubated 24 h at 37° C. before drug addition (niraparib 5 μM with or without AsiDNA 2.504). Cells were harvested on day 7 after treatment, stained with 0.4% trypan blue (Sigma Aldrich, Saint-Louis, USA) and counted under microscope using Kova slides. Cell survival was calculated as the ratio of living treated cells/living not-treated cells. Cell death was calculated as the number of dead cells out of the total number of counted cells. Cells are then washed to remove drugs, and again seeded in 6-well culture plates for recovery during 6 days. A second cycle of treatment/recovery was then started. Four cycles were performed. (FIG. 4A).

Results

Conventional anticancer treatments, and more recently targeted therapies, have improved the control of tumors, but side effects that limit dose escalation and the onset of resistance during treatment, are the prime causes of therapy failure. Under the selective pressure of anti-cancer treatments, resistant populations of cancer cells invariably evolve giving rise to “resistant clones” that have adapted to the new environment induced by the treatment.

In this context, we evaluated the effect of AsiDNA co-treatment on acquired resistance to the PARP inhibitor niraparib onset.

Development of poly (adenosine disphosphate [ADP]-ribose) polymerase inhibitors (PARPi) to treat patients with inefficient HR repair have been the first example of exploiting DNA repair defects to treat cancer. However, mechanisms of resistance to this only FDA-approved family of DNA repair inhibitors have been described in preclinical models and in clinic (Chiarugi A. Trends Pharmacol Sci. 2012 January; 33(1):42-8). SKOV-3 cells, have been treated with repeated cycles of niraparib (5 μM) (High doses corresponding to the IC90) with or without AsiDNA (low dose—2.5 μM). Three independent populations of each treatment have been grown and maintained during four cycles of treatment (FIG. 4A). Clear resistance has been observed to talazoparib in all the independent populations (FIG. 4B). Interestingly, two cell populations treated with both niraparib and AsiDNA, remains very sensitive to the drugs demonstrating that AsiDNA, at a low sub-active dose, abrogated the emergence of acquired resistance to niraparib. These results strongly suggest that AsiDNA could be used in combination with niraparib for ovarian cancer treatment to delay or abrogate niraparib acquired resistance onset.

### Example 5: AsiDNA Abrogates the Emergence of Resistance to Talazoparib in Small Cell Lung Cancer

Materials and Methods

Cell Culture

The small cell lung cancer (SCLC) cell line NCI-H446 was grown according to the supplier's instructions, in RPMI medium supplemented with 10% FBS and maintained in a humidified atmosphere at 37° C. and 5% CO₂.

Drug Treatment and Measurement of Cellular Survival

For repeated cycles of treatment protocol, the drug cytotoxicity was measured by relative survival and cell death quantification. Cells were seeded in 6-well culture plates at appropriate densities and incubated 24 h at 37° C. before drug addition. Cells were harvested on day 7 after treatment, stained with 0.4% trypan blue (Sigma Aldrich, Saint-Louis, USA) and counted under microscope using Kova slides. Cell survival was calculated as the ratio of living treated cells/living not-treated cells. Cell death was calculated as the number of dead cells out of the total number of counted cells. Cells are then washed to remove AsiDNA, and again seeded in 6-well culture plates for recovery during 6 days. A second cycle of treatment/recovery was then started. Four cycles were performed. (FIG. 5A).

Results

Conventional anticancer treatments, and more recently targeted therapies, have improved the control of tumors, but side effects that limit dose escalation and the onset of resistance during treatment, are the prime causes of therapy failure. Under the selective pressure of anti-cancer treatments, resistant populations of cancer cells invariably evolve giving rise to “resistant clones” that have adapted to the new environment induced by the treatment.

In this context, we evaluated the effect of AsiDNA co-treatment on acquired resistance to PARP inhibitor onset.

Development of poly (adenosine disphosphate [ADP]-ribose) polymerase inhibitors (PARPi) to treat patients with inefficient HR repair have been the first example of exploiting DNA repair defects to treat cancer. However, mechanisms of resistance to this only FDA-approved family of DNA repair inhibitors have been described in preclinical models and in clinic (Chiarugi A. Trends Pharmacol Sci. 2012 January; 33(1):42-8). NCI-H446 cells, initially very sensitive to the PARPi talazoparib, have been treated with repeated cycles of talazoparib (100 nM) (High doses corresponding to the IC90) with or without AsiDNA (low dose—2.504). Three independent populations of each treatment have been grown and maintained during four cycles of treatment (FIG. 5A). Clear resistance has been observed to talazoparib in all the independent populations (FIG. 5B). Interestingly, cell populations treated with both talazoparib and AsiDNA, remains very sensitive to the drugs demonstrating that AsiDNA, at a low sub-active dose, abrogated the emergence of acquired resistance to talazoparib. These results strongly suggest that AsiDNA could be used in combination with PARP inhibitors for cancer therapies to delay or abrogate PARP inhibitor acquired resistance onset.

### Example 6: AsiDNA Abrogates the Emergence of Resistance to Carboplatin in SCLC

Materials and Methods

Cell Culture

Small cell lung cancer cell line NCI-H446 was grown according to the supplier's instructions, in RPMI medium supplemented with 10% FBS and maintained in a humidified atmosphere at 37° C. and 5% CO₂.

Drug Treatment and Measurement of Cellular Survival

For repeated cycles of treatment protocol, the drug cytotoxicity was measured by relative survival and cell death quantification. Cells were seeded in 6-well culture plates at appropriate densities and incubated 24 h at 37° C. before drug addition. Cells were harvested on day 7 after treatment, stained with 0.4% trypan blue (Sigma Aldrich, Saint-Louis, USA) and counted under microscope using Kova slides. Cell survival was calculated as the ratio of living treated cells/living not-treated cells. Cell death was calculated as the number of dead cells out of the total number of counted cells. Cells are then washed to remove AsiDNA, and again seeded in 6-well culture plates for recovery during 6 days. A second cycle of treatment/recovery was then started. Five cycles were performed. (FIG. 6A).

Results

Conventional anticancer treatments, and more recently targeted therapies, have improved the control of tumors, but side effects that limit dose escalation and the onset of resistance during treatment, are the prime causes of therapy failure. Under the selective pressure of anti-cancer treatments, resistant populations of cancer cells invariably evolve giving rise to “resistant clones” that have adapted to the new environment induced by the treatment.

In this context, we evaluated the effect of AsiDNA co-treatment on acquired resistance to carboplatin.

NCI-H446 cells, initially sensitive to Carboplatin, have been treated with repeated cycles of carboplatin (2.504) (High dose corresponding to the IC90) with or without AsiDNA (low dose—2.5 μM). Three independent populations of each treatment have been grown and maintained during four cycles of treatment (FIG. 6A). Clear resistance has been observed to carboplatin in all the independent populations (FIG. 6B). Interestingly, cell populations treated with both carboplatin and AsiDNA, remained very sensitive to the drugs demonstrating that AsiDNA, at a low sub-active dose, abrogated the emergence of acquired resistance to carboplatin. These results strongly suggest that AsiDNA could be used in combination with carboplatin for cancer therapies to delay or abrogate platinium salt acquired resistance onset.

1-15. (canceled)

16. A method for delaying development of cancer resistant to a cancer therapy agent in a patient having cancer comprising administering an effective amount of a Dbait molecule and, optionally, a second cancer therapy agent to the patient, thereby delaying development of cancer resistant to a cancer therapy agent.

17. The method according to claim 16, wherein the cancer therapy agent is a Dbait molecule.

18. The method according to claim 16, wherein the second cancer therapy agent is a chemotherapy or a targeted therapy.

19. The method according to claim 16, wherein the second cancer therapy agent is a PARP inhibitor.

20. The method according to claim 19, wherein the PARP inhibitor is selected from the group consisting of olaparib, rucaparib, niraparib, talazoparib, iniparib and veliparib.

21. The method according to claim 16, wherein the second cancer therapy agent is selected from the group consisting of a platinum agent, an alkylating agent, a camptothecin, a nitrogen mustard, an antibiotic, an antimetabolite, and a vinca.

22. The method according to claim 21, wherein the second cancer therapy agent is a platinum agent.

23. The method according to claim 16, wherein the Dbait molecule is administered in at least two cycles.

24. The method according to claim 16, wherein the Dbait molecule is to be administered in combination therapy with the second cancer therapy agent.

25. The method according to claim 23, wherein the Dbait molecule and the second cancer therapy agent are administered in at least two cycles.

26. The method according to claim 25, wherein the Dbait molecule and the second cancer therapy agent are administered in combination for at least three or four cycles.

27. The method according to claim 16, wherein the Dbait molecule has one of the following formulae: 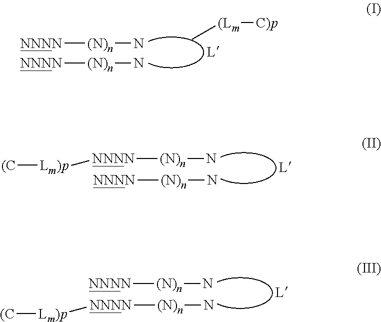
wherein N is a deoxynucleotide, n is an integer from 1 to 195, the underlined N refers to a nucleotide having or not a modified phosphodiester backbone, L′ is a linker, C is the molecule facilitating endocytosis selected from a lipophilic molecule or a ligand which targets cell receptor enabling receptor mediated endocytosis, L is a linker, m and p, independently, are an integer being 0 or 1.

28. The method according to claim 27, wherein the Dbait molecule has the following formula: 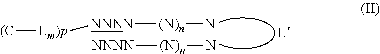
with the same definition than formulae (I), (II), and (III) for N, N, n, L, L′, C and m.

29. The method according to claim 27, wherein the Dbait molecule has the following formula:

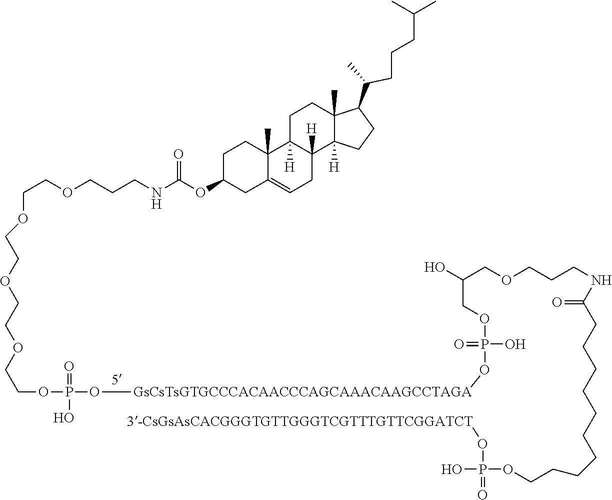

30. The method according to claim 16, wherein the cancer is selected from the group consisting of leukemia, lymphoma, sarcoma, melanoma, and cancers of the head and neck, kidney, ovary, pancreas, prostate, thyroid, lung, esophagus, breast, bladder, brain, colorectum, liver and cervix.

31. The method according to claim 22, wherein the platinum agent is selected from the group consisting of cisplatin, oxaliplatin and carboplatin.


SEQUENCE LISTING (6 sequences)

SEQ ID NO: 1 - DNA, 32 bp, artificial sequence
  Dbait32
acgcacgggtgttgggtcgtttgttcggatct

SEQ ID NO: 2 - DNA, 32 bp, artificial sequence
  Dbait32Ha
cgtaggtctgtttggtggctttgcagtggcac

SEQ ID NO: 3 - DNA, 32 bp, artificial sequence
  Dbait32Hb
gctaggcttgtttgctgggttgtaggcacagc

SEQ ID NO: 4 - DNA, 32 bp, artificial sequence
  Dbait32Hc
gctgtgcccacaacccagcaaacaagcctaga

SEQ ID NO: 5 - DNA, 32 bp, artificial sequence
  Dbait32Hd
gctaggtctgtttggtggctttgcagtggcac

SEQ ID NO: 6 - DNA, 32 bp, artificial sequence; identical to SEQ ID NO: 4
  Dbait molecule IIa
  misc_feature: at the 3′ end of the complementary strand the three last nucleotides with phosphorothioate or methylphosphonate backbone
  misc_feature (1)..(1): at the 5′ end, Lm = carboxamido oligoethylene glycol + C = single or double chain fatty acids, cholesterol, sugars, peptides or proteins
  misc_feature (1)..(1): at the 5′ end, Lm = tetraethyleneglycol + C = cholesterol
  stem_loop (1)..(32)
  modified_base (1)..(3): mod_base= phosphorothioate or methylphosphonate backbone
  misc_feature (32)..(32): loop L′ = 1,19-bis(phospho)-8-hydraza-2-hydroxy-4-oxa-9-oxo-nonadecane